(19) United States
(12) Patent Application Publication
(10) Pub. No.: US 2023/0110837 A1
(43) Pub. Date: Apr. 13, 2023

(54) METHODS OF IMPROVING QUALITY CONTROL ACCURACY FOR MEDICAL IMAGES

(71) Applicant: Alpha Intelligence Manifolds, Inc., New Taipei City (TW)

(72) Inventors: QING-ZONG TSENG, New Taipei City (TW); CHENG-WEI LIN, New Taipei City (TW); PO-YU CHEN, New Taipei City (TW)

(21) Appl. No.: 17/938,695

(22) Filed: Oct. 7, 2022

Related U.S. Application Data
(60) Provisional application No. 63/253,146, filed on Oct. 7, 2021.

(51) Int. Cl. G06T 7/00

(52) U.S. Cl. CPC G06T 7/0012; G06T 2207/30008; G06T 2207/30168; G06T 2207/20081; G06T 2207/10116

(57) ABSTRACT

The present invention relates to a method to improve quality control (QC) accuracy. The method comprises at least one input quality control (IQC) check and at least one output quality control (OQC) check for the image to be analyzed. The integrated IQC and OQC results may be mapped onto a pre-defined multi-dimensional space to evaluate the overall QC result.

### RELATED APPLICATION

This application claims the benefit of the U.S. Provisional Application No. 63/253,146 filed on Oct. 7, 2021, titled “ARTIFICIAL INTELLIGENCE DIAGNOSTIC WORKFLOW,” which is incorporated herein by reference at its entirety.

### BACKGROUND OF THE INVENTION

### Field of the Invention

The present invention relates to a method for improving the robustness of artificial intelligence (AI), especially the AI's ability to distinguish errors from good-quality results during diagnostic workflow.

### Description of Related Art

Disease diagnosis is an important part in medical field. Recent years, artificial intelligence (AI) has applied to many medical diagnostic systems to improve the efficiency and accuracy of diagnosis. Thanks to the development of neural network (NN) and deep learning (DL), an AI with desired functions can easily be trained with good performance. However, despite high accuracy, deep neural network often recognizes features different from which human vision does (arXiv:1312.6199, 2014 and arXiv:1412.1897, 2015). Also, many studies have indicated that the confidence scores are not necessarily related to probability of correctness. In other words, AI has no “feeling” of whether the result it generated is reasonable or not (arXiv:1805.11783, 2018). Therefore, unexpected errors sometimes occur even in well-trained AIs.

For AI to process medical images, object detection and classification are the main focuses. Because AI acts differently from human mental process, and because it is nearly impossible to set a decision boundary without false positives and false negatives for a classifier, diagnostic AIs occasionally produce erroneous results which are difficult to figure out where the errors come from.

In an AI performing bone image classification and disease diagnosis, an X-ray image is usually provided as an input for analysis. The image then undergoes quality check, classification, and analysis by suitable AI model(s) according to its classification, as shown in FIG. 1. If the image fails to pass the quality check or fails to be classified, the following AI model analysis is supposed to be halted or be performed with a warning. However, sometimes the AI generates the results without knowing itself making mistakes. This will generate results far from the real situations, which should always be avoided for a diagnostic AI. On the contrary, if too many criteria are applied to the quality check, it might result in excessive number of excluded input images, many of which may be with sufficient quality to be analyzed. Both above situations are which one should make utmost efforts to avoid.

Therefore, a new method to improve the robustness of AI, especially the AI's ability to distinguish erroneous inputs from good-quality inputs during diagnostic workflow, is still desired.

### SUMMARY OF THE INVENTION

To resolve the problems, the present invention provides a method to accurately perform a quality check on an input image to distinguish bad-quality inputs from good-quality inputs. Besides picking out bad-quality inputs, the present invention also provides evaluations to indicate where the bad quality originated from.

The method disclosed herein comprises: performing a plurality of quality checks on an input image by algorithms or AI models, wherein the plurality of quality checks comprises at least one input quality control (IQC) check and at least one output quality control (QOC) check; providing a pre-defined multiple-dimensional space (MDS) according to the number and type of the quality checks, wherein the dimension number of MDS is equal to or larger than the number of the quality checks; mapping the result of the quality check onto a corresponding position of the pre-defined space; and determining a QC result based on the information of the corresponding position on the pre-defined space.

In one embodiment, the IQC check comprises a view check, a region of interest (ROI) check, and a compatibility check.

In one embodiment, the OQC check comprises a landmark check.

In one embodiment for knee osteoarthritis diagnosis, the OQC check further comprises a knee joint space width (JSW) check, and a femoral-tibial angle (FTA) check.

In one embodiment for osteoporosis diagnosis, the OQC check further comprises an ROI aspect ratio check, an age check, a femoral neck width (FNI) check, a cortical thickness index (CTI) check, a CTI ratio check, and a bone mineral density (BMD) check.

In one another embodiment for osteoporosis diagnosis, the OQC check further comprises an age check and a bone mineral density (BMD) check.

Other objectives, advantages and novel features of the invention will become more apparent from the following detailed description when taken in conjunction with the accompanying drawings.

### BRIEF DESCRIPTION OF THE DRAWINGS

FIG. 1 is a block diagram of the traditional AI diagnosis QC process.

FIG. 2 is the block diagram showing the workflow of the present invention.

FIG. 3 shows a statistics of AI prediction result for 1,650 lumbar spine X-ray images. The predicted view for each column is lumbar spine with right lateral view (correct prediction), lumbar spine with left lateral view, thoracic spine with right lateral view, and thoracic spine with left lateral view, respectively.

FIG. 4A-4D shows the classification result of four input images with PA view, predicted by a trained AI model. The numbers at the top of each figure are the confidence score for the corresponding prediction. A confidence score >0.8 means the QC is passed and the prediction is accepted as true, whereas a confidence score <0.8 means the QC is not passed and the prediction is rejected.

FIG. 5 is the block diagram showing the workflow of knee joint image QC.

FIG. 6 shows an example of an X-ray image of a knee with implants. The predicted confidence score is 0.77, which means that the QC is not passed.

FIG. 7 is the block diagram showing the workflow of hip image QC.

FIG. 8 shows an example of an X-ray image of hip region with artificial joint.

FIG. 9 is the block diagram showing the workflow of spinal image QC.

FIG. 10A shows a knee joint image with points predicted by the knee landmark check model, and black circles in FIG. 10B are the range of two standard deviations for each average point. A predicted point is determined as abnormal if it exceeds the corresponding black circle.

### DETAILED DESCRIPTION OF PREFERRED EMBODIMENTS

The terminology used in the description presented below is intended to be interpreted in its broadest reasonable manner, even though it is used in conjunction with a detailed description of certain specific embodiments of the technology. Certain terms may even be emphasized below; however, any terminology intended to be interpreted in any restricted manner will be specifically defined as such in this Detailed Description section.

The embodiments introduced below can be implemented by programmable circuitry programmed or configured by software and/or firmware, or entirely by special-purpose circuitry, or in a combination of such forms. Such special-purpose circuitry (if any) can be in the form of, for example, one or more application-specific integrated circuits (ASICs), programmable logic devices (PLDs), field-programmable gate arrays (FPGAs), graphics processing units (GPUs), etc.

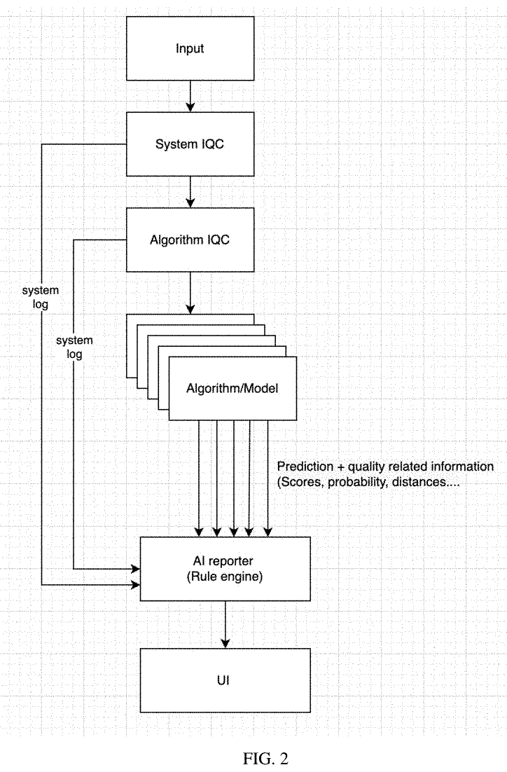

The workflow for the present application is as shown in FIG. 2. This workflow comprises system input quality control (system IQC), algorithm input quality control (algorithm IQC), model analysis and output quality control (model OQC), AI reporter (overall QC), and the output UI.

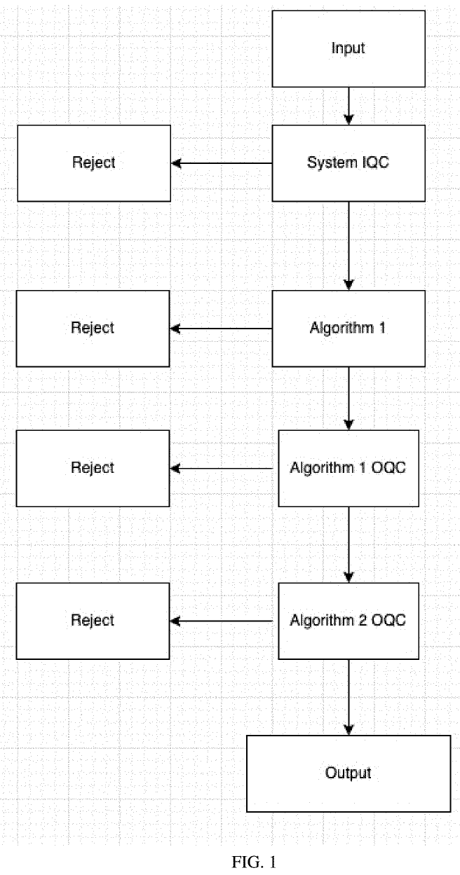

In contrast to traditional implementations, the AI reporter generates an overall QC result by analyzing all previous QC, and thus the generated overall QC is based on many different factors which may span a multi-dimensional space. Each QC may represent one or more dimensions in the multidimensional space, depending on the data acquired by the QC. For example, in QC of knee joint images, 3 IQC (View Check, ROI Check and Compatibility Check) and 3 OQC (landmark check, joint space width check and femoral-tibial angle check) may be used to assess the overall quality of the input image. Since each of the above QCs generates a numerical/true-false result, a 6-dimensional space can be constructed based on the above QC analyses. The constructed 6-dimensional space thus is able to distinguish good-quality images from bad-quality images with higher sensitivity and specificity compared to dichotomic workflow as shown in FIG. 1. In this implementation, every region in the multi-dimensional space is correlated with an overall QC result, and thereby enabling the QC result to be divided into finer classifications to provide better predictions. The classification details for each region may be made by human or may be constructed by an AI with suitable machine learning model(s). Alternatively, in the cases where the classification is not very complex, the construction of multi-dimensional space may be performed by rule sets defined by an established rule table to save the required memory. The details of the overall QC pipeline in the present application are described as below.

### 1. System IQC

The first step is to check the image format, such as size, pixel value range, color format if applicable. In the case where a Digital Imaging and Communications in Medicine (DICOM) file is the input, the related DICOM tags, such as modality type and body part examined, may also be checked. In an embodiment, the DICOM tag of the input image is checked, and the analysis workflow is determined by the body part (e.g. knee, pelvis, spine) label in the DICOM tag. In an embodiment, the analysis is aborted if the DICOM tag is missing.

### 2. Algorithm IQC

Algorithm IQC, the step after system IQC, is to use AI image analysis/recognition algorithms to check the quality of the input image. The AI algorithms may be a basic classification model trained by pre-labeled images to distinguish between good images and bad images, or it may be a classification model for specific purposes, such as distinguishing which part the image belongs to, estimating the shooting angle of the image, determining whether artificial joints and implants exist or not.

Besides classification models, a regression model may also be used to provide quality scores. One possibility is to label different images with numerical values (quality scores) during AI training.

Another alternative method is to extract the results of network operations from a certain layer in the classification model as a quality score. For example, in the last layer of a multi-class classification model, a softmax function is usually used to normalize the possibility of each category, making it sum to one, and then select the category with the highest value as the classification prediction result. This softmax score or its derived calculations can be used as the quality score. Another possibility is to extract values from the layer output before calculating softmax normalization. These values are related to the degree of activation in the network for the image under specific categories, and can also be used to calculate the quality score.

An example for algorithm IQC is training a multi-class view classifier model, which is used to determine which part of body the image belongs to, the shooting angle of the image, left or right. In addition to outputting the classification results, it also outputs the softmax value as the quality score.

In the present application, the IQC check for all types of images comprises a view check, a region of interest (ROI) check and a compatibility check. The view check is performed by a view classifier model to determine which part of body the input image belongs to. It may be executed by an AI trained by a collection of images labeled with their corresponding categories. The ROI check performs similarly but it is a little different from the view check, as the view check finds the category the whole image, and the ROI check identifies the ROI block of a specific object (e.g. a knee joint or a hip) from the input image. The ROI check may be executed by another AI trained by a collection of images labeled with a specific object and the location of ROI within the images. The compatibility check is the determine whether the classification result from the view check and the ROI check coincide with each other. While it might be considered redundant to employ both view check and ROI check to classify the input image, the application of both checks increases the accuracy of overall QC, since the AI of the two checks learns differently. The probability that both view check and ROI check provide false result is small, thus the false prediction is minimized by checking whether the two checks predict the same result.

### 3. Model OQC

This stage is to check whether the output values of the models or algorithms are reasonable and whether there is any abnormality. This can be done by setting a normal acceptable range based on the distribution of training data, a reasonable range of physiological structural measurement, or an internal value generated by the model (e.g. the maximum heatmap predicted by the landmark model at each point).

In order to complete the work of image analysis and diagnosis, the models of all IQC and OQC may be disassembled into multiple models to perform different subtasks. These subtasks usually have a sequential relationship, but is not necessarily in some cases. For example, the first model may be the object detection model to find the region of interest (bounding box) of the knee joint from the entire image. The second model may find the edge of the knee joint bone or specific anatomical locations from the region of interest (ROI) according to the results of the first model. The third model may use these points to calculate the joint space width (JSW). And the fourth model may use the points to calculate the femoral-tibial angle (FTA). The fifth model may label the areas of medial tibial, lateral tibial, medial femoral, lateral femoral and other local small areas derived from the points of previous models, and send them into models for classification or characteristic value prediction for disease classification. Examples include joint space narrowing (JSN) in degenerative arthritis, osteophyte, sclerosis, cyst, osteoporosis, bone mineral density (BMD), vertebral compression fracture and so on. Additional models may collect all or some of the above results to make new predictions. For example, a Kellegren-Lawrence grade commonly used to evaluate the severity of osteoarthritis (OA) may be predicted by the results of joint space width JSW, JSN, osteophyte, sclerosis and FTA.

### 4. AI Reporter (Overall QC)

Traditional QC processes performs a check at the output of each subtask and stop the whole process with an abnormal message if any abnormalities are found in between. In contrast, in the present application, after all models are executed, the output values of the model predictions and other auxiliary inference log (e.g. the softmax value, the deviation distance of the landmark point, etc.) are collected and used to make an integrated judgement as an overall QC result. An AI reporter integrating all predictions has several advantages, which are described below.

First, models with different functions may have misjudgments or are not robust enough to new data (such as unseen data or data from new domain). Judging the image quality based on the results of a single model has a high risk to be wrong. In contrast, different models will have a lower chance of misjudging the same data at the same time because of different task, training data, or model architecture. For example, the view classifier may misjudge the lateral view of a knee joint as a posterior-anterior view (PA view), but a PA knee object detector will not catch the object, or return a very low probability score of the bounding box, in the following step. It is almost impossible for two models to misjudge a lateral knee as PA knee at the same time.

Second, performing an integrated judgement after all the models are executed is more in line with the general process of AI debug. By doing this one can have a comprehensive overview, and is easier to find out which model has an error. Following the above example, the view classifier judges the input image as lateral view, but the object detector finds the ROI of PA view, and the landmark model shows that the prediction of some points has a large deviation compared to the average positions previously established. Combining those results together, it is very likely that the object detector gives the wrong ROI.

In the embodiments of the present application, a rule engine for the output of each model is constructed based on the previously collected test data. Some examples include whether view classifier compatible with DICOM tag, view probability, and landmark deviation. Different combinations of the output can be categorized as different groups or different positions/regions in a multi-dimensional space (MDS) for QC. The next step is to review pre-defined QC groups or QC MSD and refine the rule engine. The rule engine may be further optimized when new data comes in by iteration.

When the amount of image data is large enough, images with labeled QC tags (indicating the QC groups or the position in QC MDS) can also be use directly and combined with values generated by the models to train a QC engine AI model (meta model).

### 5. Displaying QC Results on User Interface (UI)

Last step, an AI reporter displaying QC results will combine AI results and QC rule judgment, and send back a message to the system manager to determine what kind of result and QC warning message the user will receive on the UI.

### EXAMPLES

The following examples are provided to further illustrate the image processing method as claimed.

### 1. AI Prediction Result for Images of Lumbar Spine

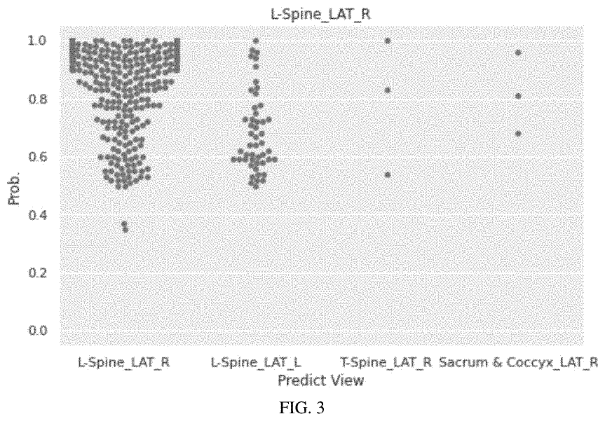

An AI is trained to distinguish more than 30 categories of X-ray bone image, each category represents a specific body part with a specific shooting method. After training, the AI is used to “predict” the categories of 1,650 input X-ray images, whose ground truth are all right lateral view of lumbar spine. The result in FIG. 3 shows that although the majority part of the inputs are predicted correctly as right lateral view of lumbar spine, a significant minority are predicted as left lateral view of lumbar spine, right lateral view of thoracic spine, or left lateral view of thoracic spine. In addition, some wrongly classified images also have high probability scores (which should indicate a high confidence), and some correctly classified images have low probability scores. Therefore, it is quite difficult to further minimize the error rate of the predicted body part by applying this single classifier.

### 2. Single ROI Check for Knee Joint Images

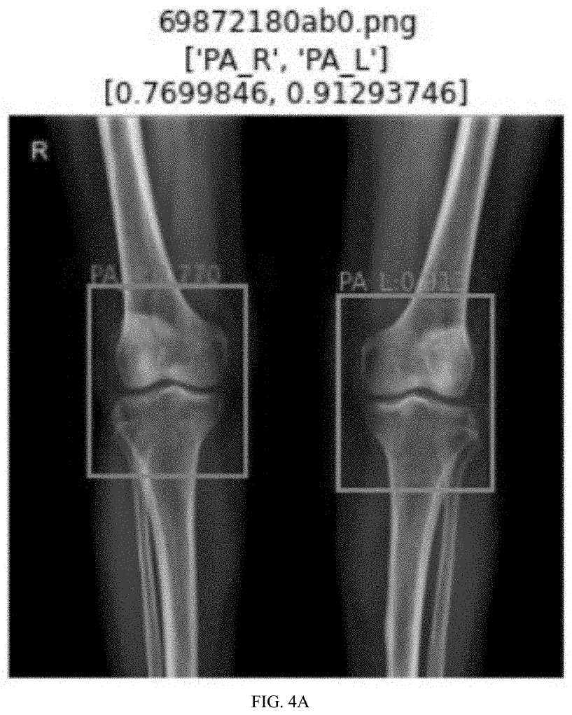

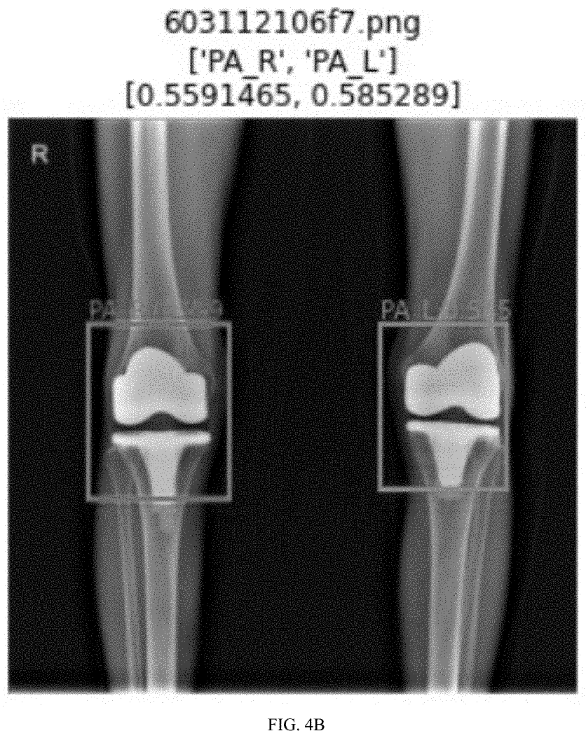

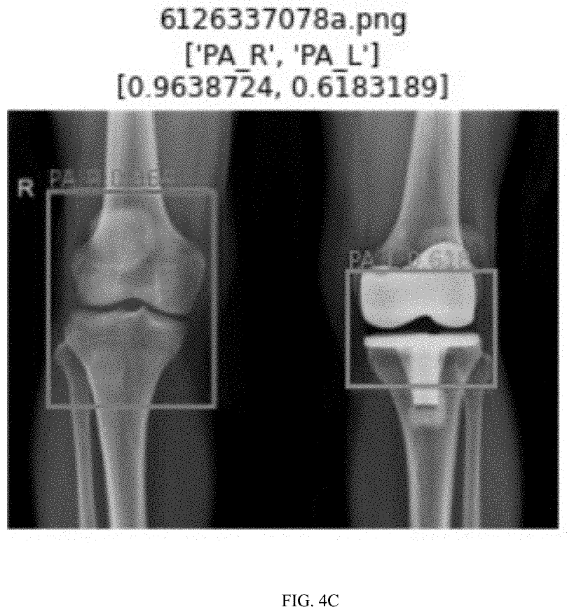

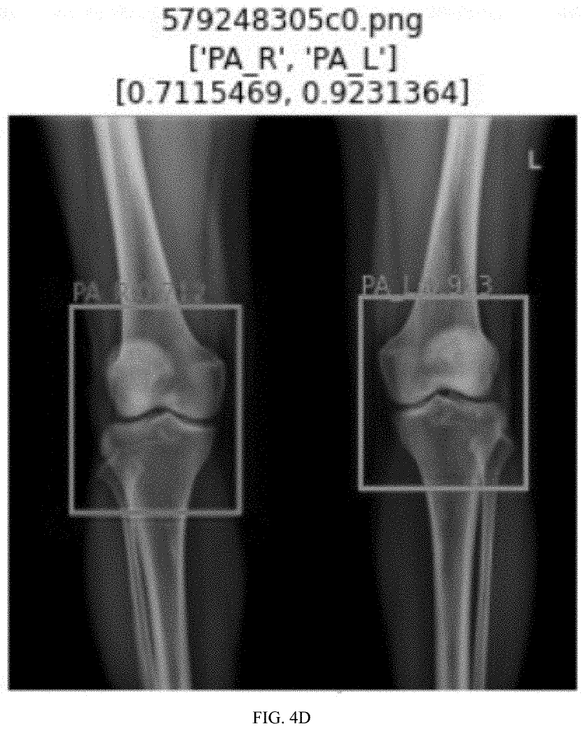

An ROI algorithm is an object detector AI trained by a set of pre-labeled data. This trained ROI check algorithm is used to judge the images of knee joints. The results of four input images with PA view are shown in FIG. 4A-4D as examples. A confidence score >0.8 indicates the classification passed the quality check. As shown in FIG. 4B and FIG. 4C, the presence of artificial joint significantly reduces the confidence score. However, it can also be observed that some normal knee joint received relatively low confidence scores, such as the right knee in FIG. 4A (0.77), and the right knee in FIG. 4D (0.71). This is another evidence showing that a single QC standard is not easy to provide satisfactory QC results.

### 3. Overall QC for Knee Joint Images

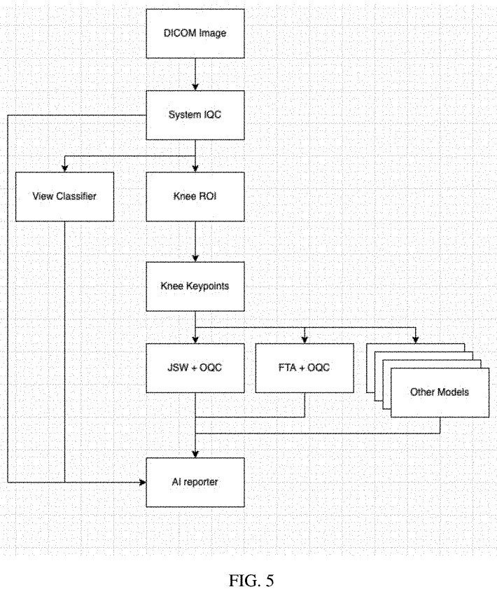

The pipeline of an overall QC for knee joint images is shown in FIG. 5. In system IQC, a DICOM image is first analyzed for relevant attributes (DICOM tags). Only files with tag “Body Part Examined” contains text string “Knee,” or “Protocol Name” contains text string “Knee” will be sent to subsequent pipeline components.

The accepted image is then analyzed by a view check (view classifier), a ROI check (knee ROI), and a compatibility check as algorithm IQC for knee joint images. The result from the Knee ROI is further fed into OQC components to perform landmark check (knee keypoints), joint space width (JSW) check and femoral-tibial angle (FTA) check. The checks for quality control of knee joint images are shown in Table 1. The details of landmark check of knee joint are provided in Example 6 below. The JSW check is performed by an AI trained to determine the distance between the apex of the medial condyle of the femur and the posterior end of the tibia (which is named joint space width) of the knee joint image. The FTA check is performed by an AI trained to measure the angle between the femur and tibia (femoral-tibial angle) of the knee joint image.

TABLE 1
The rule table for knee joint images

| | Rule Name | Rule Indicating QC Passed |
|---|---|---|
| Input | View Check | The confidence score of AI analysis > |
| Quality | | predetermined threshold value |
| Control | ROI Check | The confidence score of found ROI > |
| | | predetermined threshold value |
| | Compatibility | The analysis result of View Check |
| | Check | and ROI Check coincide with each other |
| Output | Landmark Check | The deviations of the landmark points |
| | | are within the range of averaging circles |
| Quality | JSW Check | JSW value is within a |
| | | predetermined range |
| Control | FTA Check | FTA value is within a |
| | | predetermined range |

At the overall QC stage (AI reporter), all the above results are collected and analyzed together with predefined rule sets. If none of the rules was fulfilled, the input file is considered QC pass and no warning messages will be displayed on the UI. If one or multiple QC rules were met, corresponding QC warning messages will be displayed on UI. The rule sets of knee joint QC are listed below in Table 2.

TABLE 2
The rule table of overall QC for knee joint images

| Rule No. | Rule Condition* | Details |
|---|---|---|
| 1 | IQC: | This is a good case satisfying all QC |
| | View Check = True | criteria. |
| | ROI Check = True | |
| | Compatibility Check = Ture | |
| | OQC: | |
| | Landmark Check = Ture | |
| | JSW Check = True | |
| | FTA Check = True | |
| 2 | IQC: | The ROI finder gives PA view with low |
| | ROI Check = False | confidence, and view conflict occurs, in |
| | Compatibility Check = False | addition to the absence of reasonable |
| | OQC: | landmarks. It can be inferred that this |
| | Landmark Check = False | DICOM is not with knee PA view. |
| | | The cropped ROI could be totally wrong. |
| 3 | IQC: | This is a bad case with low quality PA |
| | Compatibility Check = Ture | landmarks. Subsequent PA landmark, |
| | OQC: | JSW, DL models might not be trusted |
| | Landmark Check = False | even if they could provide results. |
| 4 | IQC: | This is a semi good example. |
| | (View Check = False) or | |
| | (ROI Check = False) or | |
| | (Compatibility Check = False) | |
| | OQC: | |
| | Landmark Check = True | |
| 5 | OQC: | This is a semi good example. The QC |
| | Landmark Check = Ture | result of JSW plot would not show the |
| | JSW Check = False | joint space width(s) which is larger than |
| | FTA Check = True | the predetermined value. |
| 6 | OQC: | This is a semi good example. There is an |
| | Landmark Check = Ture | error in measuring FTA, usually caused |
| | JSW Check = True | by tibia range too short to coordinate tibia |
| | FTA Check = False | axis. |

*If a quality control check is passed, the value = “true”, otherwise the value will be “false”.

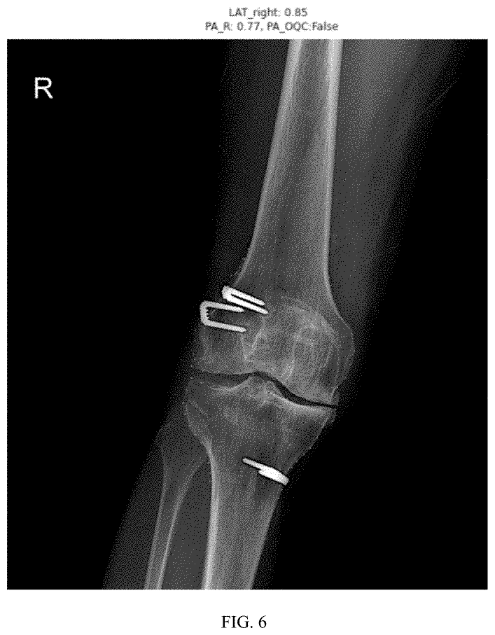

Three QC examples are given as below:
(a) An image incorrectly labeled as knee AP (anterior-poster) in DICOM tag:
1. The System IQC outputs DICOM tag “Protocol Name” as “Knees AP/PA (anterior-poster/poster-anterior) Standing”
2. the View Classifier outputs “Knee Lateral Right” with probability score >0.8,
3. the Knee ROI outputs “Knee AP/PA Right” with probability score <0.5, and
4. the Knee Keypoints outputs 5 locations with SD>2.
The AI reporter will determine this image is probably not a true knee AP image, then the UI shows a warning message of “The input image is unlikely a knee AP view.”
(b) A knee image with implant(s):
1. The System IQC outputs DICOM tag “Protocol Name” as “Knees AP Standing”
2. the View Classifier outputs “Knee AP/PA bilateral” with probability score >0.8,
3. the Knee ROI outputs “Knee AP/PA Right” with probability score <0.5, and
4. the Knee Keypoints outputs 2 locations with SD>2.
The AI reporter will determine this image is a knee AP/PA image probably with an implant or taken with non-standard imaging angle, then the UI shows a warning message of stating that the input image might contain implants or the image was taken with non-standard position.
(c) The image in FIG. 6 is a knee joint X-ray image with PA (posterior-anterior) according to its DICOM tag. However, the image is judged by the view classifier (view check) as a lateral (LAT) right knee with a score of 0.85, which is greater than the threshold >0.5 for the view classifier module. Based on the QC result, the graph will be excluded from analysis because it is not consistent with the DICOM tag.

By using the method of the present application to analyze multiple modules at the same time, the knee finder which performs ROI check will find the frontal (PA) ROI of the right knee, although the score is only 0.77 and below the threshold value 0.8, and thus will trigger the QC flag warning an abnormality. The analysis of other modules still carries out, and the output of the landmark module shows that each landmark point is within the range of the average point, and this image passes the QC criterion for landmark check. Summing below information together, one can speculate that this image should be frontal and should have implants, because the score of knee ROI check is slightly lower than the threshold value.

### 4. Overall QC for Hip Images

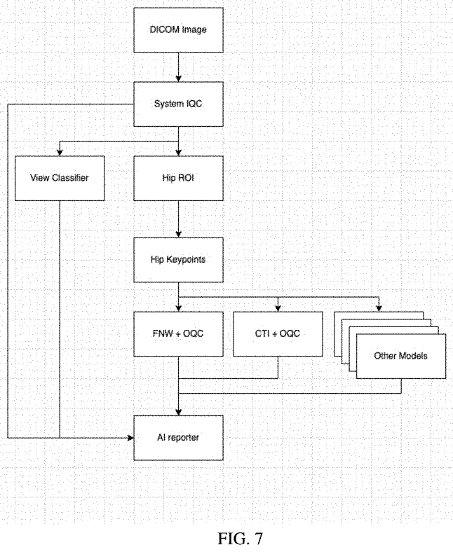

The pipeline of an overall QC for hip images is shown in FIG. 7, which is similar to the pipeline for knee joint.

The first step of the pipeline for QC of hip image is a system IQC which analyzes the DICOM tag of the input file to check whether it contains keywords such as “PELVIS”, “ABDOMEN”, etc.

In the next step, algorithm IQC, the first set of applied QC classifiers are similar to that of knee joint QC, i.e. a view check (classification of the whole image), an ROI check (finding the ROI and its category), and a compatibility check (to check whether the views determined by view check and ROI check are concordant). Using the results of the view check and its confidence value (the output value of one of the hidden layers), and the results of the ROI check (finding the ROI and its category) and confidence value, the input quality can be comprehensively judged, and a corresponding UI message can be provided to the user.

The third step, model OQC, comprises several QC methods which are specific for hip region. ROI aspect ratio check is to check whether the found ROI aspect ratio is within a specific range; hip landmark check is a landmark check to find landmark points, each point has its corresponding confidence value threshold to be satisfied to pass the QC; age check is to check whether an age information is attached on the DICOM file; FNW check is to determine whether the measured femoral neck width (FNW) is within a reasonable range; and CTI check is to determine whether the measured cortical thickness index (CTI) is within a reasonable range. The checks for quality control of hip images are as shown in Table 3. The details of landmark check of hip are provided in Example 7 below. The ROI aspect ratio check is the ratio of height to width for the ROI identified by the ROI check. The age check is to check the availability of age information on DICOM tag. The FNW check is performed by an AI trained to measure the width of femoral neck. The CTI check and the CTI ratio check are performed by AI trained to calculate the cortical thickness index and the ratio thereof, respectively.

TABLE 3
The rule table for hip images

| | Rule Name | Rule Indicating QC Passed |
|---|---|---|
| Input | View Check | The confidence score of AI analysis > |
| Quality | | predetermined threshold value |
| Control | ROI Check | The confidence score of found ROI > |
| | | predetermined threshold value |
| | Compatibility Check | The analysis result of View Check |
| | | and ROI Check coincide with each |
| | | other |
| Output | Landmark Check | The landmark confidence score > |
| Quality | | predetermined threshold values |
| Control | ROI Aspect Ratio* | The aspect ratio of ROI is within a |
| | Check | predetermined range |
| | Age Check | Age value >0 |
| | FNW Check | FNW value is within a |
| | | predetermined range |
| | CTI Check | CTI value is within a |
| | | predetermined range |
| | CTI Ratio Check | CTI ratio >0 |

*ROI aspect ratio = height/width = (yₘₐₓ − yₘᵢₙ)/(xₘₐₓ − xₘᵢₙ) for ROI

An AI reporter then collects and analyzes all the results together and provides an overall QC result with predefined rule sets, as shown in Table 4.

TABLE 4
The rule table of overall QC for hip images

| Rule No. | Rule Condition* | Details |
|---|---|---|
| 1 | IQC: | This is a good case satisfying all QC |
| | View Check = True | criteria. |
| | ROI Check = True | |
| | Compatibility Check = Ture | |
| | OQC: | |
| | Landmark Check = Ture | |
| | Age Check = True | |
| | FNW Check = Ture | |
| | CTI Check = True | |
| | CTI Ratio Check = True | |
| 2 | IQC: | The input might not be an image with hip |
| | View Check = True | PA view |
| | Compatibility Check = False | |
| 3 | IQC: | This image is too different from the AI |
| | Compatibility Check = False | model's training domain. The prediction |
| | OQC: | (if any) might not be reliable. This might |
| | Landmark Check = False | be caused by too short femoral length. |
| 4 | [IQC: | This image is too different from the AI |
| | (View Check = False) or | model's training domain. The prediction |
| | (ROI Check = False) or | (if any) might not be reliable. This might |
| | (Compatibility Check = False)] | be caused by the presence of artificial |
| | or | joint/implant or epiphyseal plate. |
| | [OQC: | |
| | Landmark Check = False] | |
| 5 | OQC: | The image does not contain the patient's |
| | Age Check = False | age when this image is captured. The lack |
| | | of age information would result in a |
| | | failure for analysis requires age |
| | | information. |
| 6 | OQC: | This image is too different from the AI |
| | (CTI Check = False) or | model's training domain. The prediction |
| | (CTI Ratio Check = False) | (if any) might not be reliable. This might |
| | | be caused by the presence of artificial |
| | | joint/implant or a too short femoral |
| | | length. |
| 7 | OQC: | This image is too different from the AI |
| | FNW Check = False | model's training domain. The prediction |
| | | (if any) might not be reliable. This might |
| | | be caused by the presence of artificial |
| | | joint/implant or epiphyseal plate. |

*If a quality control check is passed, the value = “true”, otherwise the value will be “false”.

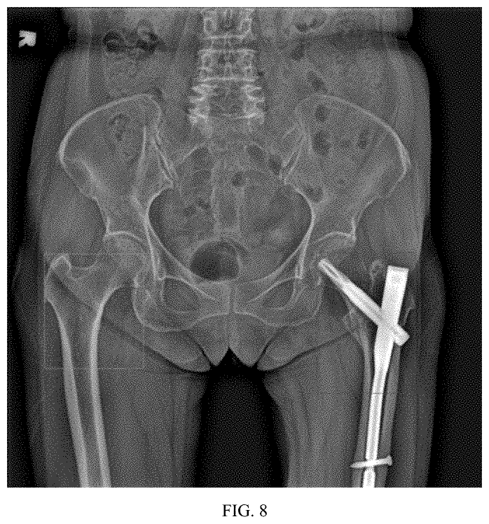

The following is an example of the overall QC for a hip image. An image of hip region with an implant (as shown in FIG. 8) is used as input image. The view classifier (view check) determines it as pelvis AP view with score=1 (greater than threshold 0.54); the hip ROI finder (ROI check) determines it as hip ROI with score=0.90 (less than threshold 0.93); the hip ROI aspect ratio check shows that the ROI is within the fit range (1.63); the hip landmark check shows that the sixth point of the hip landmark is less than the threshold (0.271<0.3), and the scores of all other points meet the threshold; the CTI check shows that the predicted value for CTI is within the normal range (0.521); and the FNW check shows the predicted value of FNW is also within the normal range (31.137).

The above result is in line with the third criterion of the hip rule engine, and the overall QC may judge that there should be an artificial joint in the image. If the QC is performed step by step without integrating all results together, the image will be kicked off in the ROI check with only a QC failed message, instead of going further and finding out that there may be implants.

### 5. Overall QC for Spinal Images

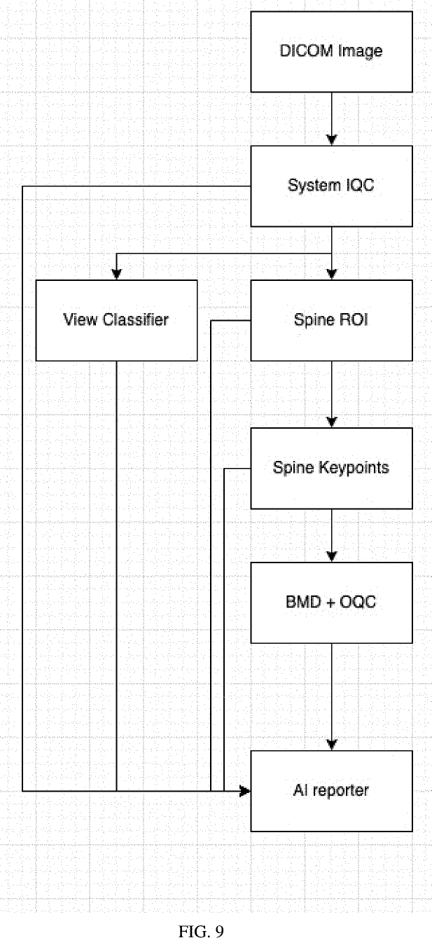

The pipeline of an overall QC for hip images is shown in FIG. 9, which is similar to the pipelines for knee joint and hip region. The IQC checks also contain a view check (classification of the whole image), an ROI check (finding the ROI and its category), and a compatibility check (to check whether the views determined by view check and ROI check are concordant); the OQC checks contain a landmark check, an age check, and a bone mineral density (BMD) check. The checks for quality control of spinal images are as shown in Table 5. The age check is to check the availability of age information on DICOM tag. The BMD check is performed by an AI trained to estimate the bone mineral density of the bone in the image, or it may also be directly imported if the BMD value is already measured in previous examination.

TABLE 5
The rule table for spinal images

| | Rule Name | Rule Indicating QC Passed |
|---|---|---|
| Input | View Check | The confidence score of AI analysis > |
| Quality | | predetermined threshold value |
| Control | ROI Check | The confidence score of found ROI > |
| | | predetermined threshold value |
| | Compatibility Check | The analysis result of View Check |
| | | and ROI Check coincide with each |
| | | other |
| | Landmark Check | The landmark score >0 |
| | Age Check | Age value >0 |
| Output | BMD Check | The BMD value is within a |
| Quality | | predetermined range |
| Control | | |

An AI reporter then collects and analyzes all the results together and provides an overall QC result with predefined rule sets, as shown in Table 6. The passed QC checks may be designated as “true”, “1”, or “positive”, whereas the unpassed QC checks may be designated as “false”, “0”, or “negative” in the multi-dimensional space.

TABLE 6
The rule table of overall QC for spinal images

| Rule No. | Rule Condition* | Details |
|---|---|---|
| 1 | IQC: | This is a good case satisfying all QC |
| | View Check = True | criteria. |
| | ROI Check = True | |
| | Compatibility Check = Ture | |
| | OQC: | |
| | Landmark Check = Ture | |
| | BMD Check = True | |
| 2 | IQC: | The input might not be an image with |
| | View Check = True | spine lateral view |
| | Compatibility Check = False | |
| 3 | IQC: | There is a concern in QC result. The |
| | (View Check = False) or | prediction (if any) might not be reliable. |
| | (ROI Check = False) or | |
| | (Compatibility Check = False) | |
| 4 | OQC: | There is a concern in QC result. The |
| | (Landmark Check = False) or | prediction (if any) might not be reliable. |
| | (BMD Check = False) | |

*If a quality control check is passed, the value = “true”, otherwise the value will be “false”.

### 6. Landmark Check for Knee Joint Images

For each knee in the image, 74 keypoints are labeled. If those points are linked with a specified order, this set of points become a shape of the knee joint.

In training data, 1,500 labeled knees are used. Those 1,500 shapes (and thus all the keypoint sets) are aligned via rotation, scaling, shifting until the distance between each shape (each keypoint to the corresponding keypoints on other shapes) became minimum. The generated shape is called the average shape. In the domain of statistical shape model (SSM), this process is called Procrustes analysis. The 1,500 shapes after the Procrustes analysis provides a mean position and standard deviation (std) for each keypoint (i.e. each keypoint has its own mean position and standard deviation).

During the QC process, each predicted keypoint are checked to see whether its distance to the corresponding keypoint on the average shape exceeded a certain threshold (e.g. 2 standard deviation).

For example, keypoint #22 might have a mean position at (X₂₂,Y₂₂), and a standard deviation (calculated from all the keypoint #22 from the 1,500 shapes). At prediction phase, the model outputs the 74 keypoints. The QC process aligns these 74 points to the average shape obtained previously so that the distance between each point to its corresponding point on the average shape becomes minimum. The keypoint #22 after the alignment process has a coordinate of (Xp, Yp), and the QC process will calculates the distance (D₂₂) between (Xp, Yp) and (X₂₂,Y₂₂). If D₂₂ is larger than the predefined threshold (e.g. 2*std₂₂), this keypoint #22 is considered abnormal.

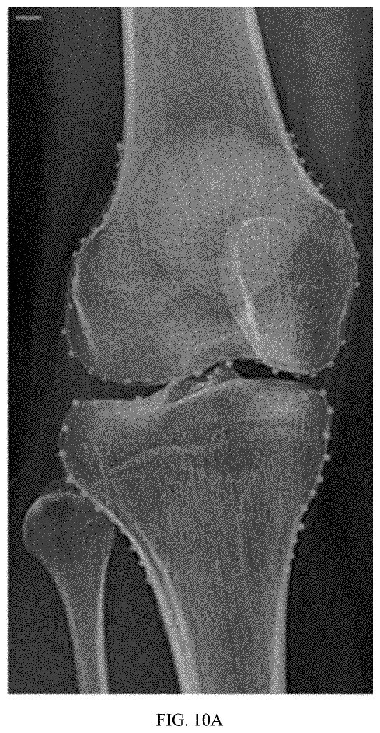

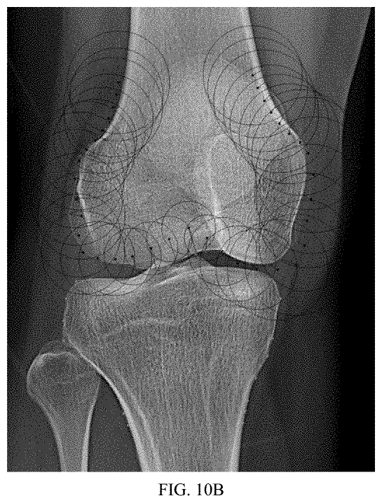

FIG. 10A shows a knee joint image with points predicted by the landmark check model, and black circles in FIG. 10B are the range of two standard deviations for each average point. A predicted point is determined as abnormal if it exceeds the corresponding black circle.

### 7. Landmark Check for Hip Images

The hip keypoint model outputs 36 keypoints (18 for each hip). Each keypoint prediction is coming from a heatmap in the last layers of the network. The predicted keypoint location is obtained from taking the location of the highest value on the heatmap (highest activation). However, in the case that the hip anatomical feature is not present (or blurred/obscured) in the input image, the heatmap will always have a highest value and thus gives nonsense prediction. The idea of the hip landmark QC process is similar to the classification model that if the activation (softmax, probability, score, etc.) is not high enough, it might be a false prediction. After analyzing the distribution of activation score for each keypoints in the training data, we can determine a set of thresholds for those 36 keypoints.

The hip keypoint model will output both the location of the keypoint as well as the corresponding scores. The QC process checks the score of each keypoint with the predefined thresholds. (e.g. keypoint #3 could have a threshold score of 0.4, while keypoint #10 could have a threshold of 0.8)

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. The claimed subject matter set forth in the claims is not intended to be limited to the embodiments shown herein but is to be accorded the widest scope consistent with the principles and novel features disclosed herein. It is contemplated that additional embodiments are within the spirit and true scope of the disclosed subject matter. Thus, it is intended that the present invention covers modifications and variations that come within the scope of the appended claims and their equivalents.

What is claimed is:

1. A method to improve quality control (QC) accuracy, comprising:
performing a plurality of quality checks on an input image by algorithms or AI models, wherein the plurality of quality checks comprises at least one input quality control (IQC) check and at least one output quality control (QOC) check;
providing a pre-defined multiple-dimensional space (MDS) according to the number and type of the quality checks, wherein the dimension number of MDS is equal to or larger than the number of the quality checks;
mapping the result of the quality check onto a corresponding position of the pre-defined space; and
determining a QC result based on the information of the corresponding position on the pre-defined space.

2. The method of claim 1, wherein the IQC check comprises a view check, a region of interest (ROI) check, and a compatibility check.

3. The method of claim 2, wherein the OQC check comprises a landmark check.

4. The method of claim 3, wherein the input image is a skeletal image of the knee region, and the input image is for knee osteoarthritis diagnosis.

5. The method of claim 4, wherein the OQC check further comprises a knee joint space width (JSW) check, and a femoral-tibial angle (FTA) check.

6. The method of claim 5, wherein a first knee QC region is the region of the MSD whose quality checks are all positive.

7. The method of claim 5, wherein a second knee QC region is the region of the MSD that the ROI check, the compatibility check and the landmark check of which are all negative.

8. The method of claim 5, wherein a third knee QC region is the region of the MSD that the landmark check of which is positive, and at least one of the view check, the ROI check and the compatibility check of which is negative

9. The method of claim 5, wherein a fourth QC region is the region of the MDS that both the landmark check and the FTA check of which are positive, and the JSW check of which is negative.

10. The method of claim 5, wherein a fifth QC region is the region of the MDS that both the landmark check and the JSW check of which are positive, and the FTA check of which is negative.

11. The method of claim 3, wherein the input image is a skeletal image of the hip region, and the input image is for osteoporosis diagnosis.

12. The method of claim 11, wherein the OQC check further comprises an ROI aspect ratio check, an age check, a femoral neck width (FNI) check, a cortical thickness index (CTI) check, a CTI ratio check, and a bone mineral density (BMD) check.

13. The method of claim 12, wherein a first hip QC region is the region of the MSD whose quality checks are all positive.

14. The method of claim 12, wherein a second hip QC region is the region of the MSD that the view check of which is positive, and the compatibility check of which is negative.

15. The method of claim 12, wherein a third hip QC region is the region of the MSD that both the compatibility check and the landmark check of which are negative.

16. The method of claim 12, wherein a fourth hip QC region is the region of the MSD that at least one of the view check, the ROI check, the compatibility check and the landmark check of which is negative.

17. The method of claim 12, wherein a fifth hip QC region is the region of the MSD that the age check of which is negative.

18. The method of claim 12, wherein a sixth hip QC region is the region of the MSD that both the CTI check and CTI ratio check of which are negative.

19. The method of claim 12, wherein a seventh hip QC region is the region of the MSD that the FNI check of which is negative.

20. The method of claim 3, wherein the input image is a skeletal image of the spine region, and the input image is for osteoporosis diagnosis.

21. The method of claim 20, wherein the OQC check further comprises an age check and a bone mineral density (BMD) check.

22. The method of claim 21, wherein a first spine QC region is the region of the MSD whose quality checks are all positive.

23. The method of claim 21, wherein a second spine QC region is the region of the MSD that the view check of which is positive, and the compatibility check of which is negative.

24. The method of claim 21, wherein a third spine QC region is the region of the MSD that at least one of the view check, the ROI check, and the compatibility check of which is negative.

25. The method of claim 21, wherein a fourth spine QC region is the region of the MSD that at least one of the age check and the BMD check of which is negative.
